(19) United States
(12) Patent Application Publication
(10) Pub. No.: US 2018/0071214 A1
(43) Pub. Date: Mar. 15, 2018

(54) Pharmaceutical Oral Dose Formulation and Composition of Matter

(71) Applicant: David W. Thrower, Boca Raton, FL (US)

(72) Inventor: David W. Thrower, Boca Raton, FL (US)

(21) Appl. No.: 15/694,949

(22) Filed: Sep. 4, 2017

Related U.S. Application Data
(60) Provisional application No. 62/394,891, filed on Sep. 15, 2016.

(51) Int. Cl. A61K 9/08; A61K 9/00; A61K 47/22; A61K 47/26; A61K 47/10; A61K 31/475; A61K 31/12; A61K 31/05; A61K 31/7048; A61K 31/451; A61K 38/13; A61K 31/436; A61K 31/17

(52) U.S. Cl. CPC A61K 9/08; A61K 9/0053; A61K 47/22; A61K 47/26; A61K 47/10; A61K 31/475; A61K 31/17; A61K 31/05; A61K 31/7048; A61K 31/451; A61K 38/13; A61K 31/436; A61K 31/12

(57) ABSTRACT

A method of formulating low solubility, low permeability and/or p-glycoprotein efflux transporter substrate drugs or herbal extracts for increased intestinal absorption and a composition of matter for said drugs or herbal extracts. The method utilizes a water, alcohol or organic solvent to complex a low solubility, low permeability and/or p-glycoprotein efflux transporter substrate drug or herbal extract, the p-glycoprotein inhibitors quercitin and piperine, with a carbohydrate. The result is a microemulsion of the drug or herbal extract which has both higher solubility and permeability than if dosed alone; as well as resistance to p-glycoprotein mediated efflux, once absorbed into the intestinal lumen.

### FIELD AND BACKGROUND OF THE INVENTION

It has been suggested by many pharmaceutical companies that an oral delivery system is a preferred method of administering therapeutic remedies; it has been estimated that some 85% of all drugs are delivered orally. The ability to deliver a drug orally, allows for increased compliance with the treatment regimen versus an intravenous delivery or even a suppository, which may cause the patient discomfort or subject them to an infection. Other treatments such as inhalation and dermal patches, allow for increased patient compliance versus an intravenous dose, but may require an excess drug product in order to be effective, due to absorption issues and usually requires a either expensive equipment or a formulation which is costly to manufacture, test, and administer. Further, oral formulations can be administered without the public noticing while in a public location such as an office or restaurant, where with most other formulations, this may not be the case. The delivery system through which a drug is ingested plays a significant role in the way and the extent to which the compound is metabolized. Problems of miscibility and absorption are numerous with such systems. In order to receive the full therapeutic benefit, the drug must be effectively absorbed into the blood stream through the intestine. However, the body possess numerous mechanisms to prevent absorption and expelling a drug back into the intestine before it can reach its site of action. Therein lies the problem, many drug compounds are poorly soluble in an aqueous medium, as found in the stomach, nor will they effectively cross the lumen of the intestine for absorption into the blood stream.

Chief among these mechanisms which prevent absorption through the intestinal wall is that of the p-glycoprotein. The p-glycoprotein is an ATP-powered efflux pump which can transport hundreds of structurally unrelated hydrophobic amphipathic compounds, including therapeutic drugs, peptides and lipid-like compounds. This polypeptide plays a crucial physiological role in protecting tissues from toxic xenobiotics and endogenous metabolites, and also affects the uptake and distribution of many clinically important drugs. It forms a major component of the blood-brain barrier, the intestinal lining's defenses against xenobiotics, and the renal tubules mechanisms to accelerate the excretion of many xenobiotics. This restricts the uptake of drugs from the intestine.

As a guide for predicting the intestinal absorption of drug substances, the U.S. Food and Drug Administration utilizes the Biopharmaceutical Classification System (BCS) to classify drug substances, including small molecule pharmaceuticals and nutraceuticals, based on both permeability and solubility. This four tiered system differentiates drugs based on their solubility and permeability. BCS Class I drug substances are both highly permeable and highly soluble. BCS Class II drug substances are highly permeable, but have low solubility. BCS Class III drug substances are highly soluble, but have low permeability. BCS Class IV drug substances have both low solubility and permeability.

There are many BCS Class III and IV drug substances which would be effective therapies for a variety of diseases, but for the fact that they poorly absorb or will not absorb through the intestine.

A first example, include the chemotherapeutic agent, vinblastinee, which must delivered intravenously, due to negligible intestinal absorption and its status as a p-glycoprotein substrate. After intravenous injection, vinblastinee, is known to cause severe tissue irritation and even damage, which further limits its utility as a chemotherapeutic agent.

A second example involves curcumin, a compound found in turmeric extract, is a known COX-II inhibitor, prophylactic chemotherapeutic agent, histamine release inhibitor and nutritional antioxidant; however, due to p-glycoprotein catalyzed efflux <1%, of an oral dose is absorbed through the intestine. Co-administration of curcumin with the p-glycoprotein inhibitor, piperine, will only increase absorption to approximately 2.4%. The low absorption requires a larger dose of curcumin, in order to reach therapeutic concentrations in the blood stream, which makes it inconvenient for the patient to self-administer and causes an unpalatable after taste, as well as GI discomfort, discoloration of the mouth and feces.

A third example involves cannibidiol (CBD), the non-psychoactive agent found in cannabis species including industrial hemp and marijuana. CBD is also found in flax seed oil, and can be legally extracted from this source. CBD is a probable anxiolytic, anticonvulsive, antispasmodic, and antipsychotic drug. It may also be useful in hospital emergency departments to relieve the dysphoric and combative symptoms associated with accidental or deliberate ingestions of some hallucinogenic drugs such as marijuana, synthetic cannabinoid (bath salts), and potentially as an antagonist of NMDA receptors such as dextromethorphan, and agonist of 5HT-2a receptors such as mescaline and lysergic acid diethyl amide (LSD). It is approved in Canada as an anticonvulsive for pediatric use in in the treatment of a severe myoclonic epilepsy of infancy and early childhood, known as Dravet syndrome. However, at only 3% bioavailability, CBD is poorly absorbed through the intestinal lumen, despite having structural characteristics of a highly absorbent molecule. It is believed that CBD's interaction and inhibition of the p-glycoprotein efflux transporter is the cause of the poor absorption; thus, by blocking this interaction, it will be possible to increase the bioavailability of a CBD oral dose.

The instant invention is a novel formulation technology which allows for low solubility, low permeability and/or p-glycoprotein efflux transporter substrate drugs and herbal extracts, such as vinblastine, curcumin, and CBD to be absorbed effectively through the intestinal lumen with lowered p-glycoprotein catalyzed efflux while emulsifying it to compensate for problems with solubility. This technique concurrently addresses three formulation problems in the following manners, while the mechanism also aids absorption: 1) inhibition of intestinal p-glycoprotein, mitigating efflux of substrates from enterocytes back into the intestinal lumen; 2) facilitation of mechanisms of absorption that circumvent the p-glycoprotein's reach; 3) this technique places the drug in a microemulsion on contact with water or intestinal chyme mediated by the amphipathic nature of the carbohydrate used, which has mutual affinity for the drug and the water; this compensates for poor solubility in water; and additionally 4) the drug may be drawn across membranes with the carbohydrate/alcohol formulation by means of hydrophobic affinity. The instant invention, therefore, satisfies the need for a drug substance formulation method which allows for the preparation of drug substances for oral administration, which would otherwise be ill-suited for oral dosage.

### SUMMARY OF THE INVENTION

There exists many drug substances and herbal extracts which are thought to have therapeutic or other health benefits, but are not currently given as oral dosages because they have low systemic bioavailability. This is due to having low solubility and/or low permeability, which prevents intestinal uptake; and/or they are substrates for the p-glycoprotein efflux transporter, which transports the drug substance or herbal extract back into the intestine, before it can reach systemic circulation, become bioavailable and reach the receptors needed to elicit a desired clinical effect. The numbers of these drug substances and herbal remedies which have low solubility, low permeability and/or are p-glycoprotein substrates, are too numerous to detail here; however, examples include: vinblastinee, curcumin, cannibidiol (CBD), digoxin, loperamide, cyclosporine, tacrolimus and talinolol.

The present invention relates to a process for formulating low solubility, low permeability and/or p-glycoprotein efflux inhibited drug substance and herbal extracts for use as oral dosages. Specifically, the present invention complexes the drug substance or herbal extract with piperine, and quercetin in a carbohydrate substrate to create a soluble mixture, using water, alcohol, ex. ethanol, or an organic solvent, such as heptane or acetone, to bring these active ingredients and the carbohydrate into solution. The resulting mixture is highly soluble and will resist removal from the intestinal lumen via the p-glycoprotein efflux transporter.

### DETAILED DESCRIPTION

Pharmaceutical ingredient or plant material is provided. These pharmaceutical ingredients or plant materials can be of any material which is considered biopharmaceutics classification system (BCS) class III or IV, i.e. low permeability and high or low solubility and/or is a substrate for the p-glycoprotein efflux transporter.

The following details the preferred embodiment for this process of composing this drug delivery formulation, involving the use of water, alcohol, or organic solvent, such as water, ethanol, diethyl ether, dimethyl ether, petroleum ethers, ethyl acetate, acetone, hexane, iso-octane (2,2,4-trimethyl pentane), or other suitable solvent, hereinafter “solvent”. The API or herbal extract, piperine or bioperine and quercitin (hereinafter, “components”), are separately dissolved in aliquots of the solvent, creating a slurry or solution; and vacuum filtered through a suitable apparatus such as a Buchner or Hirsch funnel with a suitable filter, such as a commercially available 0.5 μm nylon filter. Second, a water soluble carbohydrate excipient, such as glycerin, xylitol, sucrose, dextrose, potato starch, corn starch, or other suitable base, pre-treated with solvent, is added to each filtered component slurry or solution. The mixture is then homogenized by stirring and the reaction is allowed to proceed until it reaches the desired concentration of component and carbohydrate excipient, as determined by UV/Vis or other suitable analytical method such as HPLC and/or GC, and the carbohydrate excipient is fully dissolved. The slurry is vacuum filtered, if necessary, using a suitable apparatus such as a Buchner or Hirsch funnel with a suitable filter, such as a commercially available 0.5 μm nylon filter. The solvent is then removed from the slurry by vacuum evaporation at less than 80° C. from each of these slurries forming three batches of crystals: (API:carbohydrate), (piperine:carbohydrate), and (quercitin:carbohydrate). The three crystal types (API:carbohydrate), (piperine:carbohydrate), and (quercitin:carbohydrate) are blended, as needed, to standardize the concentration of API or herbal extract in the solution to a desired level while homogenizing the mixture. The final product may be treated with additional ingredients, such as vegetable oils, waxes, lecithin, fats, semi-solid or liquid polyols, as needed to balance the ingredients, standardize and/or stabilize the product.

Throughout the process, the API or herbal extract, piperine, or bioperine and quercitin are monitored for their concentration levels. The methodology for measuring the concentration levels may change from herbal extract to herbal extract or API to API as appropriate per compendial analytical practices, such as found in the USP guidelines. A trained natural products or pharmaceutical chemist develops the methodology or uses accepted reference methodologies.

Suitable capsules are available from many sources, and sizes from “000” to “3” are preferably used. Suitable encapsulation equipment is available from market suppliers such as Shionogi. Air can be eliminated from the capsules using an inert gas such as nitrogen, argon, helium, or other inert gases.

In an alternative embodiment, the API or herbal extract, piperine and quercitin are combined, in a single aliquot of solvent, prior to addition of the carbohydrate excipient. The process is otherwise the same as detailed above.

### EXAMPLES

The following specific examples are provided to afford a better understanding of the present invention to those skilled in the art. It is to be understood that these samples are intended to be illustrative only and are not intended to limit the invention in any way.

### Example 1

Preparation of Vinblastine Oral Modified Release with Quercitin, Piperine and Sucrose Excipients with an Ethanol Solvent:
a. Raw vinblastine is dried via vacuum drying at a temperature of <40° C. at a pressure of less than 50 mm Hg, if necessary.
b. Dissolve 1.25 g of dried vinblastine in 200 mL 95% ethanol at a PH of 9.8 at standard temperature and pressure.
c. Dissolve 2 g of piperine in 50 mL 95% ethanol at a PH of 9.8 at standard temperature and pressure.
d. Dissolve 50 g of quercitin in 150 mL 95% ethanol at a PH of 9.8 at standard temperature and pressure.
e. Prepare a 0.667 g/mL w/v solution of sucrose, using 150 g of sucrose and 225 mL 95% ethanol at standard temperature and pressure.
f. Agitate the slurries (separately) from steps (b)-(d) at 50 RPM for 5 minutes using a vertical homogenizer.
g. Vacuum filter (separately) each of the raw vinblastine, piperine, quercitin and sucrose solutions through either a Buchner's or Hirsch's funnel with a 0.5 μm nylon filter.
h. To the vinblastine, piperine and quercitin solutions, add 45 mL, 60 mL and 120 mL, respectively 0.667 g/mL sucrose slurry from (e) above.
i. Homogenize the vinblastine, piperine and quercitin/sucrose slurries at 500 RPM.
j. While under homogenization, add 3.5 L, 4.45 L and 8.9 L additional 95% ethanol at a PH of 9.8, to the vinblastine, piperine, and quercitin/sucrose slurries, respectively; allow the homogenization to continue until the solids are fully dissolved into the solution.
k. Remove any ethanol or water through vacuum evaporation at a temperature of <40° C. at a pressure of less than 50 mm Hg, while under constant homogenization at 500 RPM.
l. Grind the resultant crystals to a powder by homogenization at 2000 RPM.
m. Analyze in-process materials per compendial practices to determine the concentration of the active constituents in terms of milligram vinblastine, piperine or quercitin per gram of sucrose.
n. Calculate the ratio of grams of (vinblastine:sucrose) to (piperine:sucrose) to (quercetin:sucrose) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
o. Blend materials for a final amount of 1.25 mg vinblastine, 2 mg of piperine, 50 mg quercetin and 200 mg sucrose base, per commercially available size 3 Shionogi capsule or equivalent.
p. Analyze the final product using compendial methods to verify that the product conforms to final specifications.

### Example 2

Preparation of Curcumin Oral Modified Release with Quercitin, Piperine and Xylitol Excipients with an Ethanol Solvent:
a. Dry curcumin or a botanical material containing curcumin via vacuum drying at a temperature of <40° C. at a pressure of less than 50 mmHg, if necessary.
b. Prepare an 8.33 g/L w/v slurry of curcumin in anhydrous ethanol, with 10.0 g curcumin and 1.20 L anhydrous ethanol.
c. Prepare a 50.0 g/L w/v slurry of piperine in anhydrous ethanol, with 5.00 g of piperine and 0.100 L of anhydrous ethanol.
d. Prepare a 17.5 g/L w/v slurry of quercetin in anhydrous ethanol, with 70.0 g of quercetin and 4.00 L of anhydrous ethanol.
e. Agitate each slurry at 50 RPM for 10 minutes.
f. Separately, vacuum filtration of each slurry using a Buchner's or Hirsch funnel with a 0.5 μm nylon filter.
g. Prepare three xylitol slurries with anhydrous ethanol:
   Slurry #1: 0.688 g/mL w/v xylitol, through addition of 41.3 g xylitol to 60 mL anhydrous ethanol. This will be reserved for producing a slurry containing curcumin.
   Slurry #2: 0.690 g/mL w/v xylitol, through addition of 20.7 g xylitol to 30 mL anhydrous ethanol. This will be reserved for producing a slurry containing piperine.
   Slurry #3: 0.642 g/mL w/v xylitol, through addition of 289 g xylitol to 450 mL anhydrous ethanol. This will be reserved for producing the slurry containing quercitin.
h. Add each filtered solution from steps (b), (c) and (d) to its respective xylitol slurry from step (g).
i. Homogenize the individual curcumin, piperine and quercitin/xylitol slurries at 500 RPM.
j. While under homogenization, add 3.5 L, 1.8 L and 25 L additional 95% ethanol at a PH of 9.8, to the curcumin, piperine and quercitin/xylitol slurries, respectively; continue homogenization, until crystals are formed.
k. Remove ethanol and water through vacuum evaporation at a temperature of <40° C. and a pressure of less than 50 mmHg, while under constant homogenization at 500 RPM.
l. Grind the resultant crystals to a powder by homogenization at 2000 RPM.
m. Analyze in-process materials per compendial practices to determine the concentration of the active constituents in terms of milligram curcumin, piperine or quercitin per gram of xylitol.
n. Calculate the ratio of grams of (curcumin:xylitol) to (piperine:xylitol) to (quercetin:xylitol) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
o. Blend materials for a final amount of 10 mg curcumin, 5 mg of piperine, 75 mg of quercetin approximately 500 mg xylitol, per commercially available size 0 Shionogi capsule or equivalent.

### Example 3

Preparation of Cannibidiol (CBD) Oral Modified Release with Quercitin, Piperine and Xylitol Excipients with an Ethanol Solvent:
a. Dry CBD or a botanical material containing CBD via vacuum drying at a temperature of <40° C. at a pressure of less than 50 mmHg, if necessary.
b. Prepare a 24.4 g/L w/v slurry of CBD in 95% ethanol, with 10.0 g CBD and 410 ml of 95% ethanol, adjusted to a PH of 11.0 with sodium hydroxide.
c. Prepare a 50 g/L w/v slurry of piperine in anhydrous ethanol, with 5.00 g of piperine and 0.100 L of anhydrous ethanol.
d. Prepare a 16.7 g/L w/v slurry of quercetin in anhydrous ethanol, with 75.0 g of quercetin and 4.5 L of anhydrous ethanol.
e. Agitate each slurry at 50 RPM for 10 minutes.
f. Separately, vacuum filter each slurry using a Buchner's or Hirsch funnel with a 0.5 μm nylon filter.
g. Prepare three xylitol slurries with anhydrous ethanol:
   Slurry #1: 0.667 g/mL w/v xylitol, through addition of 40 g xylitol to 60 ml 95% ethanol at a PH of 11.0. This will be reserved for producing a slurry containing CBD.
   Slurry #2: 0.667 g/mL w/v xylitol, through addition of 20 g xylitol to 30 ml anhydrous ethanol. This will be reserved for producing a slurry containing piperine.
   Slurry #3: 0.656 g/mL w/v xylitol, through addition of 295 g xylitol tom 450 ml anhydrous ethanol. This will be reserved for producing the slurry containing quercitin.
h. Add each filtered solution from steps (b), (c) and (d) to its respective xylitol slurry from step (g).
i. Homogenize the CBD, piperine and quercitin/xylitol slurries, individually, at 500 RPM.
j. While under homogenization, add 3.3 L, 1.7 L and 25 L additional anhydrous ethanol, to the CBD, piperine and quercitin/xylitol slurries, respectively; continue homogenization, until crystals are formed.
k. Remove ethanol and water through vacuum evaporation at a temperature of <40° C., and a pressure of less than 50 mm Hg, while under constant homogenization at 500 RPM.
l. Grind the resultant crystals to a powder by homogenization at 2000 RPM.
m. Analyze in-process materials per compendial practices to determine the concentration of the active constituents in terms of milligram CBD, piperine or quercitin per gram of xylitol.
n. Calculate the ratio of grams of (CBD:xylitol) to (piperine:xylitol) to (quercetin:xylitol) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
o. Blend materials for a final amount of 10 mg CBD, 5 mg of piperine, 75 mg of quercetin approximately 500 mg xylitol, per commercially available size 0 Shionogi capsule or equivalent.

### Example 4

Preparation of Digoxin Oral Modified Release with Quercitin, Piperine and Xylitol Excipients with an Ethanol Solvent:
a. Dry digoxin or a botanical material containing digoxin, such as digitalis lantana, via vacuum drying at a temperature of <40° C. at a pressure of less than 50 mmHg, if necessary.
b. Prepare a 0.547 g/L w/v slurry of digoxin in 95% ethanol at a PH of 5, with 0.875 g digoxin and 1.6 L ml of ethanol, adjusted to a PH of 5.0 with acetic acid.
c. Prepare a 50 g/L w/v slurry of piperine in anhydrous ethanol, with 5.00 g of piperine and 0.100 L of anhydrous ethanol.
d. Prepare a 16.7 g/L w/v slurry of quercetin in anhydrous ethanol, with 50.0 g of quercetin and 3.0 L of anhydrous ethanol.
e. Agitate each slurry at 50 RPM for 10 minutes.
f. Separately vacuum filter each slurry using a Buchner's or Hirsch funnel with a 0.5 μm nylon filter.
g. Prepare three xylitol slurries with anhydrous ethanol:
   Slurry #1: 0.6 g/mL w/v xylitol, through addition of 2.4 g xylitol to 4 ml 95% ethanol at a PH of 11.0. This will be reserved for producing a slurry containing digoxin.
   Slurry #2: 0.687 g/mL w/v xylitol, through addition of 13.75 g xylitol to 20 ml anhydrous ethanol. This will be reserved for producing a slurry containing piperine.
   Slurry #3: 0.687 g/mL w/v xylitol, through addition of 137.4 g xylitol to 200 ml anhydrous ethanol. This will be reserved for producing the slurry containing quercitin.
h. Add each filtered solution from steps (b), (c) and (d) to its respective xylitol slurry from step (g).
i. Homogenize the digoxin, piperine and quercitin/xylitol slurries at 500 RPM.
j. While under homogenization, add 415 ml, 2.4 L and 23.7 L additional anhydrous ethanol, to the digoxin, piperine and quercitin/xylitol slurries, respectively; continue homogenization, until crystals are formed.
k. Remove ethanol and water through vacuum evaporation at a temperature of <40° C. and a pressure of less than 50 mmHg, while under constant homogenization at 500 RPM.
l. Grind the resultant crystals to a powder by homogenization at 2000 RPM.
m. Analyze in-process materials per compendial practices to determine the concentration of the active constituents in terms of milligrams digoxin, piperine and quercitin per gram of xylitol.
n. Calculate the ratio of grams of (digoxin:xylitol) to (piperine:xylitol) to (quercetin:xylitol) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
o. Blend materials for a final amount of 10 mg digoxin, 5 mg of piperine, 75 mg of quercetin approximately 500 mg xylitol, per commercially available size 0 Shionogi capsule or equivalent.

### Example 5

Preparation of Loperamide Oral Modified Release with Quercitin, Piperine and Glycerin Excipients with an Ethanol Solvent:
a. Dry loperamide via vacuum drying at a temperature of <40° C. at a pressure of less than 50 mmHg, if necessary.
b. Prepare an 8.0 g/L w/v slurry of loperamide in 95% ethanol, with 2.00 g loperamide and 0.25 L of 95% ethanol, adjusted to a PH of 11.0 with sodium hydroxide.
c. Prepare a 52.6 g/L w/v slurry of piperine in anhydrous ethanol, with 10.0 g of piperine and 0.190 L of anhydrous ethanol.
d. Prepare a 16.7 g/L w/v slurry of quercetin in anhydrous ethanol, with 150 g of quercetin and 8.95 L of anhydrous ethanol.
e. Agitate each slurry at 50 RPM for 10 minutes.
f. Separately, vacuum filtration of each slurry using a Buchner's or Hirsch funnel with a 0.5 μm nylon filter.
g. Add the filtered solution from step (b) to 6.8 g of glycerin. Add the filtered solution from (c) to 33.8 g of glycerin. Add the filtered solution from (d) to 507.0 g of glycerin.
h. Individually homogenize the loperamide, piperine and quercitin/glycerin slurries at 50 RPM.
i. Remove ethanol and water through vacuum evaporation at a temperature of <40° C. and a pressure of less than 50 mmHg, while under constant homogenization at 50 RPM until the slurries contains <2% ethanol by GC analysis.
j. Analyze in-process materials per compendial practices to determine the concentration of the active constituents in terms of milligram loperamide, piperine and quercitin per gram of glycerin.
k. Calculate the ratio of grams of (loperamide:glycerin) to (piperine:glycerin) to (quercetin:glycerin) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
l. Blend materials for a final amount of 2 mg loperamide, 10 mg of piperine, 150 mg of quercetin, and approximately 500 mg glycerin, per commercially available size 0 capsule.

### Example 6

Preparation of Cyclosporine Oral Modified Release with Quercitin, Piperine and Glycerin Excipients with an Ethanol Solvent:
a. Dry cyclosporine via vacuum drying at a temperature of <40° C. at a pressure of less than 50 mmHg, if necessary.
b. Prepare a 1500 g/L w/v slurry of cyclosporine in 70% ethanol with 15 g cyclosporine and 10 ml of 70% ethanol.
c. Prepare a 52.6 g/L w/v slurry of piperine in anhydrous ethanol, with 10.00 g of piperine and 190 ml of anhydrous ethanol.
d. Prepare a 5.6 g/L w/v slurry of quercetin in anhydrous ethanol, with 25.0 g of quercetin and 4.5 L of anhydrous ethanol.
e. Agitate each slurry at 50 RPM for 10 minutes.
f. Separately, vacuum filtration of each slurry using a Buchner's or Hirsch funnel with a 0.5 μm nylon filter.
g. Prepare glycerin solutions from the solutions prepared in steps (b), (c) and (d); to the filtered solution from step (b) add 76.5 g of glycerin; to the filtered solution from step (c) to 51 g of glycerin; to the filtered solution from step (d) add 382.5 g of glycerin.
h. Homogenize the cyclosporine, piperine and quercitin/glycerin slurries at 50 RPM.
i. Remove ethanol and water through vacuum evaporation at a temperature of <40° C. and a pressure of less than 50 mmHg, while under constant homogenization at 50 RPM until the slurries contains <2% ethanol by GC analysis.
j. Analyze in-process materials per compendial practices to determine the concentration of the active constituents in terms of milligram cyclosporine, piperine or quercitin per gram of xylitol.
k. Calculate the ratio of grams of (cyclosporine:glycerin) to (piperine:glycerin) to (quercetin:glycerin) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
l. Blend materials for a final amount of 15 mg cyclosporine, 10 mg of piperine, 75 mg of quercetin, and approximately 750 mg glycerin, per commercially available size 00 capsule.

### Example 7

Preparation of Tacrolimus Oral Modified Release with Quercitin, Piperine and Xylitol Excipients with an Ethanol Solvent:
a. Dry tacrolimus via vacuum drying at a temperature of <40° C. at a pressure of less than 50 mmHg, if necessary.
b. Prepare a 25.0 g/L w/v slurry of tacrolimus in 70% ethanol, with 0.25 g tacrolimus and 10 ml of 70% ethanol, adjusted to a PH of 11.0 with sodium hydroxide.
c. Prepare a 50 g/L w/v slurry of piperine in anhydrous ethanol, with 5.00 g of piperine and 0.100 L of anhydrous ethanol.
d. Prepare a 30 g/L w/v slurry of quercetin in anhydrous ethanol, with 75.0 g of quercetin and 2.5 L of anhydrous ethanol.
e. Agitate each slurry at 50 RPM for 10 minutes.
f. Separately, vacuum filtration of each slurry using a Buchner's or Hirsch funnel with a 0.5 μm nylon filter.
g. Prepare three xylitol slurries with anhydrous ethanol:
   Slurry #1: 0.70 g/mL w/v xylitol, through addition of 2.4 g xylitol to 3.4 ml of 70% ethanol. This will be reserved for producing a slurry containing tacrolimus.
   Slurry #2: 0.70 g/mL w/v xylitol, through addition of 21.7 g xylitol to 31 ml anhydrous ethanol. This will be reserved for producing a slurry containing piperine.
   Slurry #3: 0.71 g/mL w/v xylitol, through addition of 325.4 g xylitol to 460 ml anhydrous ethanol. This will be reserved for producing the slurry containing quercitin.
h. Add each filtered solution from steps (b), (c) and (d) to its respective xylitol slurry from step (g).
i. Homogenize the tacrolimus, piperine and quercitin/xylitol slurries at 500 RPM.
j. While under homogenization, add 100 ml, 1.81 L and 21.1 L additional anhydrous ethanol, to the tacrolimus, piperine and quercitin/xylitol slurries, respectively; continue homogenization, until crystals are formed.
k. Remove ethanol and water through vacuum evaporation at a temperature of <40° C. and a pressure of less than 50 mmHg, while under constant homogenization at 500 RPM.
l. Grind the resultant crystals to a powder by homogenization at 2000 RPM.
m. Analyze in-process materials per compendial practices to determine the concentration of the active constituents in terms of milligrams tacrolimus, piperine or quercitin per gram of xylitol.
n. Calculate the ratio of grams of (digoxin:xylitol) to (piperine:xylitol) to (quercetin:xylitol) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
o. Blend materials for a final amount of 250 μg tacrolimus, 5 mg of piperine, 75 mg of quercetin approximately 500 mg xylitol, per commercially available size 0 Shionogi capsule or equivalent.

### Example 8

Preparation of Talinolol Oral Modified Release with Quercitin, Piperine and Xylitol Excipients with an Ethanol Solvent:
a. Dry talinolol via vacuum drying at a temperature of <40° C. at a pressure of less than 50 mmHg, if necessary.
b. Prepare a 12.0 g/L w/v slurry of talinolol in 70% ethanol, with 15.0 g talinolol and 1.25 L of 70% ethanol, adjusted to a PH of 11.0 with sodium hydroxide.
c. Prepare a 50.0 g/L w/v slurry of piperine in anhydrous ethanol, with 10 g of piperine and 0.200 ml of anhydrous ethanol.
d. Prepare a 16.7 g/L w/v slurry of quercetin in anhydrous ethanol, with 50.0 g of quercetin and 3.00 L of anhydrous ethanol.
e. Agitate each slurry at 50 RPM for 10 minutes.
f. Separately, vacuum filtration of each slurry using a Buchner's or Hirsch funnel with a 0.5 μm nylon filter.
g. Prepare three xylitol slurries with anhydrous ethanol:
   Slurry #1: 0.697 g/mL w/v xylitol, through addition of 99.0 g xylitol to 142 ml of 95% ethanol at a PH of 11. This will be reserved for producing a slurry containing talinolol.
   Slurry #2: 0.695 g/mL w/v xylitol, through addition of 66.0 g xylitol to 95.0 ml anhydrous ethanol. This will be reserved for producing a slurry containing piperine.
   Slurry #3: 0.733 g/mL w/v xylitol, through addition of 330 xylitol to 450 ml anhydrous ethanol. This will be reserved for producing the slurry containing quercitin.
h. Add each filtered solution from steps (b), (c) and (d) to its respective xylitol slurries from step (g).
i. Individually, homogenize the talinolol, piperine and quercitin/xylitol slurries at 500 RPM.
j. While under homogenization, add 8.25 L, 5.50 L and 27.5 L additional anhydrous ethanol, to the talinolol, piperine and quercitin/xylitol slurries, respectively; continue homogenization, until crystals are formed.
k. Remove ethanol and water through vacuum evaporation at a temperature of <40° C. and a pressure of less than 50 mmHg, while under constant homogenization at 500 RPM.
l. Grind the resultant crystals to a powder by homogenization at 2000 RPM.
m. Analyze in-process materials per compendial practices to determine the concentration of the active constituents in terms of milligram talinolol, piperine or quercitin per gram of xylitol.
n. Calculate the ratio of grams of (talinolol:xylitol) to (piperine:xylitol) to (quercetin:xylitol) to any sucrose treated with the prescribed stabilizers, emollients such as vegetable oils, waxes, lecithin, fats, semi-solid and liquid polyols, antioxidants such as tocopherols, ascorbates, thymol, and anti-caking agents, as needed.
o. Blend materials for a final amount of 15 mg talinolol, 10 mg of piperine, 50 mg of quercetin approximately 750 mg xylitol, per commercially available size 00 Shionogi capsule or equivalent.

It is to be understood that the foregoing is illustrative of the present invention and is not to be construed as limited to the specific embodiments disclosed, and that modifications to the disclosed embodiments, as well as other embodiments, are intended to be included within the scope of the appended claims. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

I claim:

1. A process for providing an oral pharmaceutical formulation for a low solubility, low permeability and/or p-glycoprotein efflux transporter substrate active pharmaceutical ingredient or herbal extract, the process comprising the steps of:
a. dissolving and dispersing a pre-determined amount of said active pharmaceutical ingredient or herbal extract in a solvent; wherein said solvent is selected among a group of water, alcohol or an organic solvent;
b. dissolving and dispersing a pre-determined amount of said piperine or bioperine in a solvent; wherein said solvent is of the same type used to dissolve said active pharmaceutical ingredient or herbal extract;
c. dissolving and dispersing a pre-determined amount of said quercetin in a solvent; wherein said solvent is of the same type used to dissolve said active pharmaceutical ingredient or herbal extract;
d. dissolving and dispersing a pre-determined amount of a carbohydrate excipient in a solvent; wherein said carbohydrate excipient is a monosaccharide, disaccharide, oligosaccharide or alcohol sugar, of a chain length of no more than 21 units; wherein said solvent is of the same type used to dissolve said active pharmaceutical ingredient or herbal extract;
e. combining 1 part of said active pharmaceutical ingredient or herbal extract slurry from (a) above with an excess of said solvent, ranging from 1 to 200 parts solvent to 1 part active pharmaceutical ingredient, or as necessary to dissolve said active pharmaceutical ingredient or herbal extract and 1 to 40 parts of said carbohydrate excipient from (d) above; homogenizing until the carbohydrate excipient and active pharmaceutical or herbal extract ingredient are both dissolved in solution with the solvent;
f. combining 1 part of said piperine or bioperine slurry from (b) above with an excess of said solvent, ranging from 1 to 200 parts solvent to 1 part piperine or bioperine, or as necessary to dissolve said piperine or bioperine and 1 to 40 parts of said carbohydrate excipient from (d) above; homogenizing until the carbohydrate excipient and piperine or bioperine are both dissolved in solution with the solvent;
g. combining 1 part of said quercetin slurry from (c) above with an excess of said solvent, ranging from 1 to 200 parts solvent to 1 part quercetin, or as necessary to dissolve said quercetin and 1 to 40 parts of said carbohydrate excipient from (d) above; homogenizing until the carbohydrate excipient and quercetin are both dissolved in solution with the solvent;
h. removing said solvent from said active pharmaceutical ingredient or herbal extract, said piperine or bioperine and said quercitin slurries and drying said crystals;
i. combining said active pharmaceutical ingredient or herbal extract, said piperine or bioperine and said quercitin crystals to desired amount and dosage ratios;

2. The process of claim 1, wherein said active pharmaceutical ingredient or herbal extract is selected among a group of vinblastine, curcumin, cannibidiol, loperamide, digoxin, at least one cyclosporine, tacrolimus or talinolol.

3. The process of claim 1, wherein said solvent is water, alcohol or a water miscible organic solvent selected among a group of: water, ethanol and other alcohols, diethyl ether, dimethyl ether, petroleum ethers, ethyl acetate and other low molecular weight esters, acetone, hexane, iso-octane (2,2,4-trimethyl pentane), and other low molecular weight alkanes, or an alkyl halide.

4. The process of claim 1, wherein said carbohydrate excipient is monosaccharide, disaccharide, oligosaccharide or alcohol sugar of no more than 21 units, is selected among a group of glycerin, xylitol, mannitol, erythritol, sorbitol, sucrose, dextrose, its counter enantiomer, fructose, lactose, galactose, ribose, deoxyribose, potato starch and corn starch.

5. An oral pharmaceutical composition comprising: a) a low solubility, low permeability and/or p-glycoprotein substrate active pharmaceutical ingredient or herbal extract; b) quercitin; c) piperine; d) a carbohydrate excipient; wherein the active pharmaceutical ingredient or herbal extract, piperine and quercitin, are individually complexed with said carbohydrate at a ratio of one part active pharmaceutical ingredient or herbal extract, piperine or quercitin with up to 40 parts carbohydrate excipient.

6. The oral pharmaceutical composition of claim 5, wherein the active pharmaceutical ingredient or herbal extract is selected among a group of vinblastine, curcumin, cannibidiol, loperamide, digoxin, at least one cyclosporine, tacrolimus or talinolol.

7. The oral pharmaceutical composition of claim 5, wherein said carbohydrate excipient is monosaccharide, disaccharide, oligosaccharide or alcohol sugar of no more than 21 units, is selected among a group of glycerin, xylitol, mannitol, erythritol, sorbitol, sucrose, dextrose, its counter enantiomer, fructose, lactose, galactose, ribose, deoxyribose, potato starch and corn starch.
